(19) United States
(12) Patent Application Publication
(10) Pub. No.: US 2020/0330641 A1
(43) Pub. Date: Oct. 22, 2020

(54) BIODEGRADABLE GRAPHENE OXIDE BIOCOMPOSITE FIBROUS MEMBRANE, PREPARATION METHOD AND USES THEREOF

(71) Applicant: Shulan Jiang, Jiangmen City (CN)

(72) Inventor: Shulan Jiang, Jiangmen City (CN)

(21) Appl. No.: 16/387,071

(22) Filed: Apr. 17, 2019

(51) Int. Cl. A61L 15/18; A61L 17/10; A61L 17/12; A61L 15/22

(52) U.S. Cl. CPC A61L 15/18; A61L 17/10; A61L 17/12; A61L 17/105; A61L 2300/418; A61L 2300/10; A61L 2400/12; A61L 2300/404; A61L 15/225

(57) ABSTRACT

The invention relates to a biodegradable graphene oxide biocomposite fibrous membrane and a preparation method and uses thereof. The composite fibrous membrane comprises biodegradable graphene oxide biocomposite fibers, each fiber has an outer layer consisting of graphene oxide-biodegradable polymer nanofibers and an inner layer consisting of sodium alginate/polyvinyl alcohol nanocomposite fibers. The biodegradable graphene oxide biocomposite fibrous membrane of the invention has the advantages of good biocompatibility, biodegradability, swellability, bacteriostasis and good mechanical properties and chemical stability.

### FIELD OF THE INVENTION

The invention relates to the field of graphene oxide materials, in particular to a biodegradable graphene oxide biocomposite fibrous membrane, preparation method and uses thereof.

### BACKGROUND OF THE INVENTION

Electrospinning is a spinning method in which a polymer solution or a polymer melt is spray-stretched and finally solidified into fibers under the action of an electrostatic force. In recent years, with rapid development of nanotechnology, electrospinning technology has been further improved and developed. Nanofibers prepared by electrospinning technology have gradually gained attention in biomedicine field. The fibrous membrane prepared by electrospinning has many advantages such as high specific surface area, controllable fiber diameter, porous structure and so on, which makes this material has a good application prospect in drug delivery, wound dressing, biomimetic material, medical care and the like.

Graphene oxide has good hydrophilicity, bacteriostasis and biocompatibility, and it is easily surface functionalized due to a large number of aerobic functional groups on its surface, such as carboxyl group, carbonyl group, hydroxyl group, epoxy group and the like. Therefore, it has a broad application prospect in biomedicine field.

At present, there are some studies on the application in medical materials, but few on medical dressings, and these studies generally focused on single function improvement such as antibacterial properties, mechanical properties, etc. And there is a lack of materials that have a combination function of bacteriostatic, biocompatible, biodegradable, and better swellability. For example, Chinese patent CN 103920179 B discloses a graphene wound dressing comprising an antibacterial layer prepared by graphene. This patent mainly utilizes the bacteriostasis property of graphene. Chinese patent CN 107137753 A discloses a graphene/carbon nanofiber medical non-woven fabric, in which a non-woven fabric for carrying drug is formed by embedding graphene powder into a shell layer or a core layer of a polymer polymer fiber by using coaxial electrospinning technique. The patent mainly utilizes the two-dimensional layered structure of graphene to increase the pore volume and specific surface area, thereby increasing the ability to load drugs.

### SUMMARY OF THE INVENTION

To solve the above technical problems, the invention provides a biodegradable graphene oxide biocomposite fibrous membrane, and provides a preparation method and uses of the biodegradable graphene oxide biocomposite fibrous membrane. The aim of the invention is to provide a complex material containing graphene oxide, which has multiple advantages such as good biocompatibility, biodegradability, swelling, bacteriostatic properties, and good mechanical properties and chemical stability.

To achieve the aim of the present invention, the following technical solutions are adopted:

A biodegradable graphene oxide biocomposite fibrous membrane, which comprising biodegradable graphene oxide biocomposite fibers, each fiber has an outer layer consisting of graphene oxide-biodegradable polymer nanofibers and an inner layer consisting of sodium alginate/polyvinyl alcohol nanocomposite fibers.

Preferably, the weight ratio of graphene oxide and the biodegradable polymer in the outer layer of the graphene oxide-biodegradable polymer nanofiber is about 1: (25-100).

Preferably, the biodegradable polymer is polylactic acid (PLA), polyglycolic acid (PGA), polycaprolactone (PCL), polydioxanone (PDO) or a copolymer thereof.

Preferably, the sodium alginate in the inner layer of the sodium alginate/polyvinyl alcohol nanocomposite fibers accounts for about 10%-40% by weight based on the total weight of sodium alginate and polyvinyl alcohol.

The preparation method of the biodegradable graphene oxide biocomposite fibrous membrane comprises the following steps:
(1) ultrasonically dispersing the graphene oxide in solvent A to form a uniform graphene oxide solution; dissolving the biodegradable polymer in solvent B to form a biodegradable polymer solution;
(2) mixing the graphene oxide solution and the biodegradable polymer solution prepared in the step (1) and stirring uniformly to form an electrospinning solution C for the outer layer, wherein the weight ratio of the graphene oxide to the biodegradable polymer is about 1:(25-100);
(3) separately dissolving sodium alginate and polyvinyl alcohol in deionized water to form an aqueous solution of sodium alginate and an aqueous solution of polyvinyl alcohol, then mixing the two aqueous solutions in proportion to form an electrospinning solution D for the inner layer, wherein the sodium alginate accounts for 10%-40% by weight based on the total weight of sodium alginate and polyvinyl alcohol;
(4) adding the outer layer electrospinning solution C and the inner layer electrospinning solution D into an electrospinning machine, carrying out coaxial electrospinning and extruding the fibers from the inner tube and the outer tube into the coagulating bath of calcium chloride-ethanol solution for crosslinking for 30-60 mins, subsequently washing with absolute ethyl alcohol and vacuum drying to obtain a biodegradable graphene oxide biocomposite fibrous membrane.

Preferably, the biodegradable polymer is polylactic acid (PLA), polyglycolic acid (PGA), polycaprolactone (PCL), polydioxanone (PDO) or a copolymer thereof.

Preferably, the solvent A in the step (1) is N,N-dimethylformamide or absolute ethyl alcohol.

Preferably, the solvent B in the step (1) is dichloromethane or trichloromethane.

Preferably, the process parameters of the coaxial electrospinning are: voltage: 10 kV-20 kV, extrusion speed: 0.1-1 ml/h, horizontal distance from the needle to the receiving device: 10-20 cm, temperature: 25-30° C., humidity 50-80%.

Preferably, the weight percentage of calcium chloride in the coagulation bath of calcium chloride-ethanol solution is about 1 wt %-3 wt %.

The biodegradable graphene oxide biocomposite fibrous membrane of the invention can be used as a medical dressing, a surgical suture and drug carrier material.

The invention has the following beneficial effects:

A biodegradable graphene oxide biocomposite fibrous membrane comprises biodegradable graphene oxide biocomposite fibers, each fiber has an outer layer of graphene oxide-biodegradable polymer nanofibers and an inner layer of sodium alginate/polyvinyl alcohol nanocomposite fibers. Wherein, the outer layer comprises complex fibers which are formed by graphene oxide combined with biodegradable polymer, such as PLA, PGA, PCL or PDO, etc. In one aspect, the addition of graphene oxide may effectively increases the fiber strength, improves mechanical properties of the fibers, and simultaneously obtains nanofibers with higher porosity, higher specific surface area, excellent antibacterial property, and extremely low cytotoxicity; in another aspect, the addition of PLA, PGA, PCL or PDO may bring good biodegradability, biocompatibility and bioabsorbability to the prepared fibers; the inner layer adopts the polyvinyl alcoho/alginate composite fibers which in one hand has good biocompatibility, higher hygroscopicity, good bacteriostatic properties and good hemostatic effect by using sodium alginate, and in the other hand has good mechanical properties and chemical stability by using polyvinyl alcohol, which is suitable for using as medical materials, especially using as medical dressings. Therefore, the biodegradable graphene oxide biocomposite fibrous membrane of the present invention has the advantages of the inner layer and the outer layer, which means it has good biocompatibility, biodegradability, swelling property, bacteriostasis, hemostatic function and good mechanical properties and chemical stability and so on.

The biodegradable graphene oxide biocomposite fibrous membrane of the invention can be used as medical materials, especially as medical dressings, surgical suture, etc. When comparing with traditional medical dressings, it is more suitable for exudative wounds because of its good swellability and bacteriostatic property which can provide a good environment for regenerating healing of the wounds and reduce the risks of infection, and further it is much easier to biodegrade after use.

In addition, the biodegradable graphene oxide biocomposite fibrous membrane of the invention also has a good application prospect when using as drug carrier.

### DETAILED DESCRIPTION OF THE INVENTION

The invention is further illustrated by the following specific examples, but the protection scope of the invention is not limited by the examples.

### Example 1

The graphene oxide is ultrasonically dispersed in absolute ethyl alcohol to form a uniform graphene oxide solution; the polylactic acid is dissolved in trichloromethane to form a biodegradable polymer solution; the prepared graphene oxide solution and the biodegradable polymer solution are mixed and uniformly stirred to form an outer electrospinning solution C, wherein the weight ratio of the graphene oxide to the biodegradable polymer in the outer electrospinning solution C is 1:50; the sodium alginate and polyvinyl alcohol are seperately dissolved in deionized water to form an aqueous sodium alginate solution and an aqueous polyvinyl alcohol solution, the two aqueous solutions are mixed and stirred uniformly to form an inner layer electrospinning solution D, wherein the sodium alginate accounts for 10 wt % based on the total weight of sodium alginate and polyvinyl alcohol. Then the outer electrospinning solution C and the inner electrospinning solution D are added into an electrospinning machine for coaxial electrospinning with the process parameters are: voltage: 10 kV-20 kV, extrusion speed: 0.1-1 ml/h, the horizontal distance from the needle to the receiving device: 10-20 cm, temperature: 25-30° C., humidity 50-80%. The fibers from the inner and outer tube are extruded to the coagulating bath of calcium chloride-ethanol solution for crosslinking for 30-60 mins. The weight percentage of calcium chloride in the coagulation bath of calcium chloride-ethanol solution is 1 wt %. The resultant is washed with absolute ethanol and vacuum dried to obtain a biodegradable graphene oxide biocomposite fibrous membrane.

### Example 2

The graphene oxide is ultrasonically dispersed in anhydrous ethanol to form a uniform graphene oxide solution; the polylactic acid is dissolved in dichloromethane to form a biodegradable polymer solution; the prepared graphene oxide solution and the biodegradable polymer solution are mixed and uniformly stirred to form an outer electrospinning solution C, wherein the weight ratio of the graphene oxide to the biodegradable polymer in the outer electrospinning solution C is 1:100; the sodium alginate and polyvinyl alcohol are separately dissolved in deionized water to form an aqueous sodium alginate solution and an aqueous polyvinyl alcohol solution, the two aqueous solutions are mixed and stirred uniformly to form an inner layer electrospinning solution D, wherein the sodium alginate accounts for 30 wt % based on the total weight of sodium alginate and polyvinyl alcohol. Then the outer electrospinning solution C and the inner electrospinning solution D are added into an electrospinning machine for coaxial electrospinning with the process parameters are: voltage: 10 kV-20 kV, extrusion speed: 0.1-1 ml/h, the horizontal distance from the needle to the receiving device: 10-20 cm, temperature: 25-30° C., humidity 100-80%. The fibers from the inner and outer tube are extruded to the coagulating bath of calcium chloride-ethanol solution for crosslinking for 30-60 mins. The weight percentage of calcium chloride in the coagulation bath of calcium chloride-ethanol solution is 1 wt %. The resultant is washed with absolute ethanol and vacuum dried to obtain a biodegradable graphene oxide biocomposite fibrous membrane.

### Example 3

The graphene oxide is ultrasonically dispersed in N,N-dimethylformamide to form a uniform graphene oxide solution; the polylactic acid is dissolved in polycaprolactone to form a biodegradable polymer solution; the prepared graphene oxide solution and the biodegradable polymer solution are mixed and uniformly stirred to form an outer electrospinning solution C, wherein the weight ratio of the graphene oxide to the biodegradable polymer in the outer electrospinning solution C is 1:75; the sodium alginate and polyvinyl alcohol are separately dissolved in deionized water to form an aqueous sodium alginate solution and an aqueous polyvinyl alcohol solution, the two aqueous solutions are mixed and stirred uniformly to form an inner layer electrospinning solution D, wherein the sodium alginate accounts for 20 wt % based on the total weight of sodium alginate and polyvinyl alcohol. Then the outer electrospinning solution C and the inner electrospinning solution D are added into an electrospinning machine for coaxial electrospinning with the process parameters are: voltage: 10 kV-20 kV, extrusion speed: 0.1-1 ml/h, the horizontal distance from the needle to the receiving device: 10-20 cm, temperature: 25-30° C., humidity 100-80%. The fibers from the inner and outer tube are extruded to the coagulating bath of calcium chloride-ethanol solution for crosslinking for 30-60 mins. The weight percentage of calcium chloride in the coagulation bath of calcium chloride-ethanol solution is 1 wt %. The resultant is washed with absolute ethanol and vacuum dried to obtain a biodegradable graphene oxide biocomposite fibrous membrane.

### Example 4

The graphene oxide is ultrasonically dispersed in absolute ethyl alcohol to form a uniform graphene oxide solution; the polyglycolic acid is dissolved in dichloromethane to form a biodegradable polymer solution; the prepared graphene oxide solution and the biodegradable polymer solution are mixed and uniformly stirred to form an outer electrospinning solution C, wherein the weight ratio of the graphene oxide to the biodegradable polymer in the outer electrospinning solution C is 1:25; the sodium alginate and polyvinyl alcohol are separately dissolved in deionized water to form an aqueous sodium alginate solution and an aqueous polyvinyl alcohol solution, the two aqueous solutions are mixed and stirred uniformly to form an inner layer electrospinning solution D, wherein the sodium alginate accounts for 40 wt % based on the total weight of sodium alginate and polyvinyl alcohol. Then the outer electrospinning solution C and the inner electrospinning solution D are added into an electrospinning machine for coaxial electrospinning with the process parameters are: voltage: 10 kV-20 kV, extrusion speed: 0.1-1 ml/h, the horizontal distance from the needle to the receiving device: 10-20 cm, temperature: 25-30° C., humidity 100-80%. The fibers from the inner and outer tube are extruded to the coagulating bath of calcium chloride-ethanol solution for crosslinking for 30-60 mins. The weight percentage of calcium chloride in the coagulation bath of calcium chloride-ethanol solution is 3 wt %. The resultant is washed with absolute ethanol and vacuum dried to obtain a biodegradable graphene oxide biocomposite fibrous membrane.

The biodegradable graphene oxide biocomposite fibrous membrane of the invention has the advantages of good biocompatibility, biodegradability and bacteriostasis, which is a good medical material. It can be used as a medical dressing or a surgical suture and also has a good prospect applied as drug carrier.

That which is claimed:

1. A biodegradable graphene oxide biocomposite fibrous membrane, characterized by comprising biodegradable graphene oxide biocomposite fibers, each fiber has an outer layer consisting of graphene oxide-biodegradable polymer nanofibers and an inner layer consisting of sodium alginate/polyvinyl alcohol nanocomposite fibers.

2. The biodegradable graphene biocomposite fibrous membrane according to claim 1, wherein the weight ratio of graphene oxide and the biodegradable polymer in the outer layer of the graphene oxide-biodegradable polymer nanofibers is about 1: (25-100).

3. The biodegradable graphene oxide biocomposite fibrous membrane according to claim 1, wherein the biodegradable polymer is polylactic acid (PLA), polyglycolic acid (PGA), polycaprolactone (PCL), polydioxanone (PDO) or a copolymer thereof.

4. The biodegradable graphene oxide biocomposite fibrous membrane according to claim 1, wherein the sodium alginate in the inner layer of the sodium alginate/polyvinyl alcohol nanocomposite fibers accounts for about 10%-40% by weight based on the total weight of sodium alginate and polyvinyl alcohol.

5. The method for preparing the biodegradable graphene oxide biocomposite fibrous membrane according to claim 1, comprising the steps of:
(1) ultrasonically dispersing the graphene oxide in solvent A to form a uniform graphene oxide solution; dissolving the biodegradable polymer in solvent B to form a biodegradable polymer solution;
(2) mixing the graphene oxide solution and the biodegradable polymer solution prepared in the step (1) and stirring uniformly to form an electrospinning solution C for the outer layer, wherein the weight ratio of the graphene oxide to the biodegradable polymer is about 1:(25-100);
(3) separately dissolving sodium alginate and polyvinyl alcohol in deionized water to form an aqueous solution of sodium alginate and an aqueous solution of polyvinyl alcohol, then mixing the two aqueous solutions in proportion to form an electrospinning solution D for the inner layer, wherein the sodium alginate accounts for 10%-40% by weight based on the total weight of sodium alginate and polyvinyl alcohol;
(4) adding the outer layer electrospinning solution C and the inner layer electrospinning solution D into an electrospinning machine, carrying out coaxial electrospinning and extruding the fibers from the inner tube and the outer tube into the coagulating bath of calcium chloride-ethanol solution for crosslinking for 30-60 mins, subsequently washing with absolute ethyl alcohol and vacuum drying to obtain a biodegradable graphene oxide biocomposite fibrous membrane.

6. The method for preparing a biodegradable graphene oxide biocomposite fibrous membrane according to claim 5, wherein the biodegradable polymer is polylactic acid (PLA), polyglycolic acid (PGA), polycaprolactone (PCL), polydioxanone (PDO) or a copolymer thereof.

7. The method for preparing a biodegradable graphene oxide biocomposite fibrous membrane according to claim 5, wherein the solvent A in the step (1) is N,N-dimethylformamide or absolute ethyl alcohol.

8. The method for preparing a biodegradable graphene oxide biocomposite fibrous membrane according to claim 5, wherein the solvent B in the step (1) is dichloromethane or trichloromcthane.

9. The method for preparing a biodegradable graphene oxide biocomposite fibrous membrane according to claim 5, wherein the weight percentage of calcium chloride in the coagulation bath of calcium chloride-ethanol solution is about 1 wt %-3 wt %.

10. Use of the biodegradable graphene oxide biocomposite fibrous membrane as medical dressing, surgical sutures, or drug carrier.

11. A method of preparing a medical dressing, surgical sutures, or drug carrier, the method comprising using the biodegradable graphene oxide biocomposite fibrous membrane of claim 1.
